(19) United States
(12) Patent Application Publication
(10) Pub. No.: US 2012/0219644 A1
(43) Pub. Date: Aug. 30, 2012

(54) COCONUT OIL-BASED INTRANASAL COMPOSITION AND USE

(76) Inventors: Carolyn J. Harrington, Pittsford, NY (US); Robert A. Harrington, Pittsford, NY (US)

(21) Appl. No.: 13/406,373

(22) Filed: Feb. 27, 2012

Related U.S. Application Data
(60) Provisional application No. 61/464,111, filed on Feb. 28, 2011.

(51) Int. Cl. A61K 36/889; C11C 3/00; A61P 29/00; A61P 31/00; A61P 11/02

(52) U.S. Cl. 424/727; 554/1

(57) ABSTRACT

The present invention relates to the use of coconut oil as an active agent in natural, health-promoting products, particularly for intranasal use.

### CROSS REFERENCE TO RELATED APPLICATIONS

Priority is claimed to: U.S. Provisional Patent Application Ser. No. 61/464,111 by C. Harrington et al., entitled “COCONUT OIL-BASED INTRANASAL COMPOSITION AND USE”, filed on Feb. 28, 2011, the disclosure of which is incorporated herein by reference.

### FIELD OF THE INVENTION

The present invention relates to the use of coconut oil as an active agent in natural, health-promoting products, particularly for intranasal use.

### BACKGROUND OF THE INVENTION

Humanity suffers from innumerable ills, among them those of respiratory origin. Increases in world population, lack of proper diet, and rapid travel between distance regions and high concentration of individuals in confined areas where there is poor air quality have resulted in the increase in the number of people suffering from respiratory ailments.

The onset of respiratory difficulties is primarily a result of inhalation of airborne material through the nose and mouth. The oral cavity is better equipped to handle airborne materials, such as pathogens, before they can enter the body. Saliva in the mouth captures many airborne materials and stomach acids are highly effective in handling foreign materials before they can enter the body. The nasal passages, on the other hand, are less effective in trapping and killing microorganisms. Airborne materials inhaled through the nose usually enter the lungs and potentially enter the blood stream.

Allergy refers to any condition of the body mounting an attack on a specific foreign substance. People can experience allergic reactions to foods, chemicals, plants, animals and a variety of airborne substances. The symptoms of allergic diseases, such as allergic rhinitis (hay fever), allergic dermatitis, and allergic asthma, can be caused by a variety of allergens, such as grasses, trees, weeds, animal dander, insects, molds, drugs, dust mites, pollen, dust, airborne pollution, and chemicals. The typical symptoms of allergic rhinitis include sneezing, itchy nose, nasal congestion or inflammation, and runny nose, often accompanied by watery and itchy eyes, and postnasal drip.

The world is full of microorganisms, and so are people's bodies—in and on the skin, in the gut, and in other orifices. Friendly bacteria are vital to proper development of the immune system, to protection against microorganisms that could cause disease, and to the digestion and absorption of food and nutrients. Each person's mix of bacteria varies. Interactions between a person and the microorganisms in his body, and among the microorganisms themselves, can be crucial to the person's health and well-being. A healthy system with balanced microflora can boost the immune system, keeping pathogenic diseases in check. Unfriendly microorganisms can also upset the balance, triggering the body's immuno-response to many allergens.

Various methods have been utilized to deal with nasal-related conditions, particularly congestion and inflammation. Pharmaceutical agents for infections and allergic diseases have been developed and used. These agents may be provided in the form of sprays, vapor rubs, moisturizers, pills, capsules, liquids, etc. Many such pharmaceutical agents have adverse effects.

### Problem to be Solved

As a result, there remains a need for antimicrobial and anti-allergic agents derived from natural products, where long-term administration is possible, safety is high, no adverse reaction takes place, and which enhance the inherent protective mechanisms in the nose. In short, there remains a need for a healthier option to combat nasal inflammation and congestion with fewer side effects.

The present invention describes the use of coconut oil for intranasal application to promote good health and for treatment and/or prevention of disease and other adverse conditions, particularly nasal inflammation and congestion.

### SUMMARY OF THE INVENTION

The present invention relates to a natural composition comprising coconut oil applied to the intranasal cavity. The present invention relates to a natural composition comprising coconut oil, cayenne pepper, lemon balm, and grapefruit seed extract applied to the intranasal cavity. The present invention also relates to a method of providing a healthy intranasal space comprising applying a small amount of an intranasal composition to the tip of a cotton swab, placing the thumb and index finger on the swab stem, placing the cotton tip of the swab just inside of the nostril opening, and using a gentle motion, applying the composition to the surface of the nasal cavity, wherein said intranasal composition comprises coconut oil, as well as a method of balancing intranasal microflora comprising applying an intranasal composition to the surface of the nasal cavity, wherein said intranasal composition comprises coconut oil, as well as a method of balancing intranasal microflora comprising applying an intranasal composition to the surface of the nasal cavity, wherein said intranasal composition comprises coconut oil.

### Advantageous Effect of the Invention

The present invention includes several advantages, not all of which are incorporated in a single embodiment. The present compositions promote a healthy nose and nasal cavity. The disclosed compositions provide relief from unhealthy nasal conditions, especially those related to unbalanced microbial growth, which can lead to congestion and inflammation. The compositions may have antimicrobial properties, particularly antibacterial and antiviral properties.

### DETAILED DESCRIPTION OF THE INVENTION

The present invention relates to a composition comprising coconut oil for topical administration. More specifically, the present disclosure provides natural compositions comprising coconut oil as an active ingredient for promoting a healthy intranasal space. The present inventive composition may contain additional active ingredients having natural, homeopathic, medicinal, pharmaceutical, and/or cosmetic properties. In addition, the present invention relates to methods of using these compositions to promote good health of the intranasal space as well as alleviate unhealthy conditions and any related symptoms in the intranasal space.

As used herein, the terms “active ingredient” and “active agent” refer to ingredients that produce a natural, homeopathic, medicinal, pharmaceutical or cosmetic effect.

The term “nasal cavity,” refers to all aspects of the nasal cavity, including the nostrils, nasal/mucus membrane, cilia, and sinuses.

The term “intranasal space” refers to the special cavity inside the nose.

Coconut oil is recognized as a valuable natural product that promotes health in a variety of ways. Coconut oil is useful as a nutritional component, skin emollient and is also known to have antimicrobial properties, antifungal and antiviral properties. Traditional usages of coconut oil can be found at www.coconutresearchcenter.org. Coconut oil is a natural source of medium chain fatty acids. Coconut oil may be unrefined or refined. Fractionated coconut oil refers to coconut oil that has been refined, for example, to comprise only the saturated fat portion of the oil by removing the long-chain triglycerides.

Coconut oil is present as the primary active ingredient in the present invention. Coconut oil may be present in the compositions in an amount ranging from 0.1-100% by weight of the composition.

Preferably, the coconut oil comprises a fractionated coconut oil. The oil is colorless and virtually odorless and contains high levels of medium chain triglycerides, Caprylic (C8) and Capric (C10). Fractionated coconut oil is liquid at room temperature and very stable.

The compositions may also comprise at least one other ingredient having natural, homeopathic, medicinal, pharmaceutical, and/or cosmetic properties. Most preferably, the other ingredients are all natural ingredients, to produce a completely natural composition. Any additional active agents may be present in an amount ranging from 0.1-99.9% by weight of the composition, for example from 0.1-50% by weight, 0.1-25% by weight, 0.1-10% by weight, 0.1-5% by weight, or even 0.1-0.9% by weight of the composition.

In a preferred embodiment, the composition includes cayenne pepper, lemon balm, grapefruit seed extract, and candelilla wax.

Cayenne pepper is a red, hot chili pepper mostly known for its use in spicy dishes, however, there are also a great many associated health benefits. Used in the west since the 17th century, it has been found to treat such things as fever, cold, diarrhea; it relieves constipation, headache, and sinus congestion. Applied externally (to tired feet, sore muscles, strains and sprains) it acts as a pain reliever through a little known numbing effect. The active ingredient, capsaicin, from which cayenne's genus name, capsicum, is derived, is responsible for both cayenne's heat as well as its medicinal function. One of the most impressive properties of cayenne pepper is its quick absorption and consequent fast action. A pinch in each nostril starts working immediately to remove excess mucous and relieve sinus congestion. A dab of capsaicin extract placed in the nose has been said to relieve headaches.

Lemon balm is used medicinally as an herbal tea, or in extract form. It is claimed to have antibacterial and antiviral properties. It is also used as an anxiolytic, mild sedative or calming agent. Lemon balm and preparations thereof also have shown to improve mood and mental performance. The extract of lemon balm was also found to have exceptionally high antioxidant activity. Lemon balm contains eugenol, which kills bacteria and has been shown to calm muscles and numb tissues. It also contains tannins that contribute to its antiviral effects, as well as terpenes that add to its soothing effects.

Grapefruit seed extract has been used as a sedative, and as an antispasmodic. There is evidence that the extract of citrus grapefruit seed has antioxidant properties. Analysis shows the constituents of the seed extract and pulp are flavonoids, ascorbic acid (commonly known as vitamin C), tocopherols, citric acid, limonoids, sterols, and minerals.

The compositions of the present invention may also contain other pharmaceutically and/or cosmetically acceptable ingredients, including, but not limited to, emollients, moisturizers, conditioners, antioxidants, fillers, dyes and other coloring agents, preservatives, emulsifying agents, surfactants, stabilizers, thickeners, such as, for example, silicone materials, and other materials known in the art.

A variety of additional ingredients can be incorporated into the composition of the present invention. Non-limiting examples of these additional ingredients include vitamins and derivatives thereof (e.g. tocopherol, panthenol), thickening agents, saturated and/or unsaturated alkyl alpha hydroxy acids, resins, gums, waxes (both naturally occurring and synthetic), polymers, abrasive scrub particles, preservatives, skin penetration aids, skin bleaching (or lightening) agents, chelators and sequestrants, and aesthetic components such as essential oils, skin sensates, astringents, skin soothing agents, skin healing agents. Non-limiting examples of these aesthetic components include aloe vera, pantothenic acid and its derivatives, clove oil, menthol, camphor, eucalyptus oil, eugenol, menthyl lactate, witch hazel distillate, allantoin, bisabalol, and dipotassium glycyrrhizinate.

Examples of suitable emollients include, but are not limited to, volatile and non-volatile silicone oils (e.g., dimethicone, cyclomethicone, dimethiconol, and the like), highly branched hydrocarbons, and mixtures thereof, sunflower (Helianthus annuus) oil, canola (Brassica napus/campestris) oil, sweet almond (Prunus amygdalus dulcis) oil, sesame (Sesamum indicum) oil, and macadamia (Macadamia ternifolia) nut oil. The emollients can typically comprise in total from about 0.1 percent to about 25 percent, more preferably from about 0.5 percent to about 10 percent, and most preferably from about 0.5 percent to about 5 percent by weight of the composition.

The composition may also comprise a suitable gelling agent, thickening or other texture modification ingredients. A preferred texturizing component is candelilla wax. Useful gelling or thickening agents may include, but are not limited to, cellulose esters such as hydroxypropyl cellulose, hydroxyethyl cellulose, polyvinylpyrrolidone, carboxyvinyl polymer to thicken the composition to a desired gel consistency.

The compositions are preferably in a form suitable for topical application, particularly as an ointment for application to the nasal cavity. Although the preferred embodiment is an ointment for intranasal application, other forms may be useful, such as emulsions, suspensions, creams, lotions, gels, liquids, solids, and sprays.

The present composition is intended to provide an antimicrobial function, as well as anti-inflammatory and decongestant properties. The present composition also provides prebiotic-type or probiotic-type properties through balanced microflora in the intranasal space.

On the skin, the biggest chemical barrier to infectious organisms is the acid layer of the skin. Healthy skin has a pH of 5, making it slightly acidic. Our sweat and body oils promote this acidic environment. Harmless bacteria can tolerate the acid and live on the skin, but troublesome bacteria can't thrive and their numbers are few. Sebum present on the skin contains medium chain fatty acids, in the form of medium chain triglycerides, that can be released to fight harmful organisms. At least one type of bacterium normally present on the skin feeds on the sebum, breaking down the triglycerides into free fatty acids. The bacteria feed on the glycerol part of the triglyceride, freeing the fatty acids from the glycerol unit. Medium chain fatty acids have no immediate antimicrobial properties, when bound to glycerol units, as they are in coconut and palm kernel oils. Freed fatty acids, however, have antimicrobial properties. The combination of the slightly acidic skin pH and the medium chain fatty acids provides a protective chemical layer on the skin that prevents infection from disease-causing organisms. Due primarily to the action of bacteria, the oil on the surface of the hair and skin is composed of between 40 to 60 percent free fatty acids. The medium chain fatty acids in the sebum in combination with the acidic skin pH provide the protective layer on the skin that kills harmful germs.

When medium chain triglyceride oils derived from coconut oil or palm kernel oil is put on the skin, it doesn't have immediate antimicrobial action. However, when bacteria on the skin turn these triglycerides into free fatty acids, the result is an increase in the number of antimicrobial fatty acids on the skin, resulting in protection from infection. Although the properties and conditions of the intranasal space differ from those of the skin, for example, with respect to the absence of sebum in the intranasal space and the natural pH of the skin as opposed to the properties of mucous membranes, it is believed that the bacteria present in the intranasal space also turn the triglycerides present in coconut oil into free fatty acids, resulting in an increase in the number of antimicrobial fatty acids on the skin and in protection from infection.

The present includes methods of using these compositions to promote good health and/or for treatment and/or prevention of disease. In one use, the composition is used to balance intranasal microflora by applying an intranasal composition to the surface of the nasal cavity, wherein the intranasal composition comprises coconut oil. In another use, the composition is applied to the nasal cavity as follows:

a. apply a small amount to the tip of a cotton swab;

b. place the thumb and index finger on the swab stem;

c. carefully place the cotton tip of the swab just inside of the nostril opening;

d. using a gentle motion, make 3 or 4 circles to fully apply the composition to the nasal cavity, being careful not to insert more than half way into your nasal cavity;

e. discard the swab; and

f. repeat these steps for the other nostril.

The present method is not limited to a swab or tissue-type applicator. Other applicators which may be safely inserted into the nasal cavity may be used, for example, a fingertip. For example, a small amount of the composition may be applied to a tissue, which is then carefully placed just inside of the nostril opening. Using a gentle motion, 3 or 4 circles are made with the tissue to fully apply the composition to the nasal cavity, and the tissue is discarded, followed by the repetition of these steps for the other nostril.

The invention has been described in detail with particular reference to certain preferred embodiments thereof, but it will be understood that variations and modifications can be effected within the spirit and scope of the invention.

1. A natural composition comprising coconut oil applied to the intranasal cavity.

2. The composition of claim 1 wherein said coconut oil is fractionated coconut oil.

3. The composition of claim 1 wherein said composition provides antimicrobial properties.

4. The composition of claim 1 wherein said composition provides decongestant or anti-inflammatory properties.

5. The composition of claim 1 wherein said composition provides prebiotic-type or probiotic-type properties through balanced microflora.

6. The composition of claim 1 further comprising at least one of cayenne pepper, lemon balm, and grapefruit seed extract.

7. The composition of claim 6 further comprising candelilla wax.

8. A method of providing a healthy intranasal space comprising:
a. applying a small amount of an intranasal composition to the tip of a cotton swab;
b. placing the thumb and index finger on the swab stem;
c. placing the cotton tip of the swab just inside of the nostril opening; and
d. using a gentle motion, applying the composition to the surface of the nasal cavity, wherein said intranasal composition comprises coconut oil.

9. A method of balancing intranasal microflora comprising:
a. applying an intranasal composition to the surface of the nasal cavity, wherein said intranasal composition comprises coconut oil.
